(19) United States
(12) Patent Application Publication
(10) Pub. No.: US 2025/0138126 A1
(43) Pub. Date: May 1, 2025

(54) METHODS AND DEVICES FOR MAGNETIC RESONANCE IMAGING

(71) Applicant: SHANGHAI UNITED IMAGING HEALTHCARE CO., LTD., Shanghai (CN)

(72) Inventors: Jun YANG, Shanghai (CN); Naijia LIU, Shanghai (CN); Zhenkui WANG, Shanghai (CN)

(73) Assignee: SHANGHAI UNITED IMAGING HEALTHCARE CO., LTD., Shanghai (CN)

(21) Appl. No.: 18/932,921

(22) Filed: Oct. 31, 2024

(30) Foreign Application Priority Data
Oct. 31, 2023 (CN) 202311436793.7
Dec. 25, 2023 (CN) 202311800252.8

(51) Int. Cl. G01R 33/56; A61B 5/055; G01R 33/563; G06T 11/00

(52) U.S. Cl. CPC G01R 33/5608; G01R 33/56308; G06T 11/005; A61B 5/055; G06T 2210/41; G06T 2211/424; G06T 2211/441

(57) ABSTRACT

Methods and devices for magnetic resonance imaging are provided in embodiments of the present disclosure. The method may include determining, based on undersampling K-space data of a plurality of phases of an object, a first coil sensitivity map, the undersampling K-space data being obtained through Cartesian sampling, determining, based on the first coil sensitivity map and the undersampling K-space data, a plurality of intermediate reconstruction images, each of the plurality of the intermediate reconstruction images corresponding to one of the plurality of phases, and determining, based on the plurality of intermediate reconstruction images, an optimized dynamic image of the object through optimization processing.

### CROSS-REFERENCE TO RELATED APPLICATIONS

This application claims priority of Chinese Patent Application No. 202311436793.7 filed on Oct. 31, 2023, and Chinese Patent Application No. 202311800252.8 filed on Dec. 25, 2023, the entire contents of each of which are incorporated herein by reference.

### TECHNICAL FIELD

The present disclosure relates to the field of medical imaging, and in particular, to methods, devices, and computer-readable storage mediums for magnetic resonance imaging.

### BACKGROUND

Magnetic resonance imaging (MRI) is a widely used medical imaging technique. In order to improve the imaging speed of MRI, scan data (e.g., K-space data) is usually collected by undersampling K-space. However, the undersampling K-space data may affect the imaging quality of MRI. Therefore, it is desired to provide methods and devices for MRI to improve the imaging quality of image reconstruction performed using undersampling K-space data.

### SUMMARY

One aspect of the present disclosure provides a method for magnetic resonance imaging. The method may be implemented on a device including one or more processors and one or more storage devices. The method may include determining, based on undersampling K-space data of a plurality of phases of an object, a first coil sensitivity map, the undersampling K-space data being obtained through Cartesian sampling, determining, based on the first coil sensitivity map and the undersampling K-space data, a plurality of intermediate reconstruction images, each of the plurality of intermediate reconstruction images corresponding to one of the plurality of phases, and determining, based on the plurality of intermediate reconstruction images, an optimized dynamic image of the object through optimization processing.

Another aspect of the present disclosure provides at least one storage device including a set of instructions, and at least one processor in communication with the at least one storage device. When executing the set of instructions, the at least one processor may be directed to perform operations including: determining, based on undersampling K-space data of a plurality of phases of an object, a first coil sensitivity map, the undersampling K-space data being obtained through Cartesian sampling; determining, based on the first coil sensitivity map and the undersampling K-space data, a plurality of intermediate reconstruction images, each of the plurality of intermediate reconstruction images corresponding to one of the plurality of phases; and determining, based on the plurality of intermediate reconstruction images, an optimized dynamic image of the object through optimization processing.

Another aspect of the present disclosure provides a non-transitory computer-readable storage medium. The storage medium may include one set of instructions. When executed by one or more processors of a computing device, the at least one set of instructions may cause the computing device to perform the method.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary magnetic resonance imaging (MRI) system according to some embodiments of the present disclosure;

FIG. 2 is a schematic diagram illustrating exemplary software/hardware of a computing device according to some embodiments of the present disclosure;

FIG. 3 is a schematic diagram illustrating an exemplary processing device according to some embodiments of the present disclosure;

FIG. 4 is a flowchart illustrating an exemplary process for magnetic resonance imaging according to some embodiments of the present disclosure;

FIG. 5 is a schematic diagram illustrating an exemplary undersampling K-space data mask of a plurality of phases according to some embodiments of the present disclosure;

FIG. 6 is a schematic diagram illustrating an exemplary process for determining intermediate K-space data according to some embodiments of the present disclosure;

FIG. 7 is a schematic diagram illustrating an exemplary process for determining intermediate K-space data according to another embodiment of the present disclosure;

FIG. 8 is a schematic diagram illustrating an exemplary image reconstruction model according to some embodiments of the present disclosure;

FIG. 9 is a schematic diagram illustrating an exemplary image reconstruction model according to some embodiments of the present disclosure;

FIG. 10 is a schematic diagram illustrating an exemplary training process of an iteration module according to some embodiments of the present disclosure; and

FIG. 11 is a schematic diagram illustrating an exemplary reconstruction result according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” or “block,” as used herein, refers to logic embodied in hardware or firmware, or to a collection of software instructions. A module or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an Electrically Programmable Read-Only-Memory (EPROM). It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It may be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

The function and method of operation of these and other features, characteristics, and related structural elements of the present application, as well as component combinations and manufacturing economy, may become more apparent from the following description of the accompanying drawings, which constitute part of the specification of this application. It should be understood, however, that the drawings are for purposes of illustration and description only and are not intended to limit the scope of the present disclosure. It should be understood that the drawings are not to scale.

The terminology used herein is to describe particular example embodiments only and is not intended to be limiting. As used herein, the singular forms “a,” “an,” and “the” may be intended to include the plural forms as well, unless the context indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

The flowchart is used in this specification to illustrate the operations performed by the system according to the embodiment of the present disclosure, and the relevant description is to help better understand the magnetic resonance imaging method and/or system. It should be understood that the preceding or following operations are not necessarily performed in the exact order. Instead, the various steps can be processed in reverse order or simultaneously. At the same time, other actions can be added to these procedures, or a step or steps can be removed from these procedures.

The term “image” in the present disclosure is used to collectively refer to image data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “anatomical structure” in the present disclosure may refer to gas (e.g., air), liquid (e.g., water), solid (e.g., stone), cell, tissue, organ of a subject, or any combination thereof, which may be displayed in an image (e.g., a second image, or a first image, etc.) and really exist in or on the subject's body. The term “region,” “position,” and “region” in the present disclosure may refer to a position of an anatomical structure shown in the image or an actual position of the anatomical structure existing in or on the subject's body, since the image may indicate the actual position of a certain anatomical structure existing in or on the subject's body. The terms “organ” and “tissue” are used interchangeably referring to a portion of a subject.

During magnetic resonance imaging (MRI), to improve the imaging speed and quality, scanning data (e.g., K-space data) may be rapidly obtained through undersampling, and a reconstructed magnetic resonance image may be obtained by performing image reconstruction on the undersampling scanning data.

In some embodiments, the magnetic resonance image may be obtained by filling other data not sampled based on the sampled data in the undersampling scanning data. However, the magnetic resonance image generated in this way suffers from poor quality.

In some embodiments, the image reconstruction may be performed using the undersampling scanning data combined with a priori constraint of a neural network model. However, limited by the constraint of a count of iterations in the training process of the neural network model, the model converges poorly, which reduces the quality of the reconstruction image obtained based on the neural network model.

Embodiments of the present disclosure disclose methods and devices for magnetic resonance imaging. A first coil sensitivity map may be determined based on undersampling K-space data of a plurality of phases of at least one slice of an object; a plurality of intermediate reconstruction images corresponding to the plurality of phase phases may be determined based on the first coil sensitivity map and the undersampling K-space data; and an optimized dynamic image of the object may be determined based on the plurality of intermediate reconstruction images through optimization processing. In the method for magnetic resonance imaging, the iterative reconstruction may be performed based on the undersampling K-space data of the plurality of phases and the first coil sensitivity map, which ensures that an error between the reconstructed image and the undersampling K-space data of the plurality of phases is relatively small during the reconstruction process, that is, the reconstructed image is closer to a real multi-phase magnetic resonance image of the object, thereby improving the reconstruction quality of the magnetic resonance image.

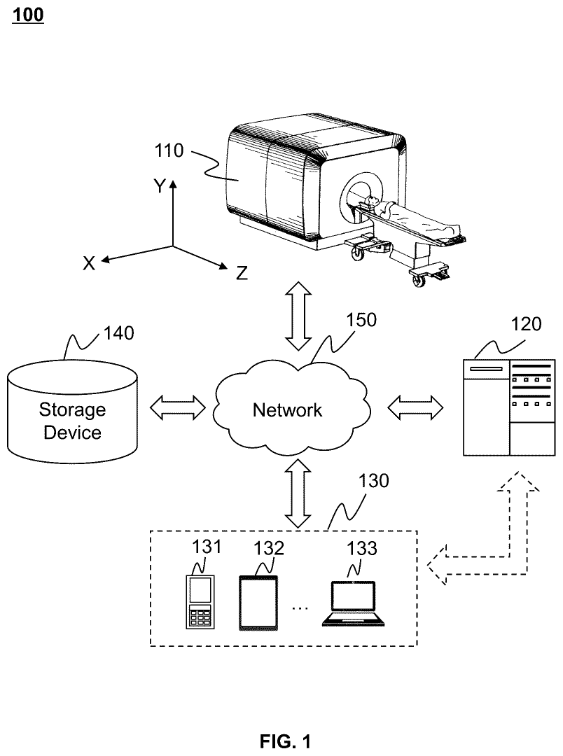

FIG. 1 is a schematic diagram illustrating an exemplary magnetic resonance imaging (MRI) system according to some embodiments of the present disclosure.

The MRI system 100 may be commonly used to obtain an interior image from a patient for a particular region of interest that can be used for the purposes of, e.g., diagnosis, treatment, or the like, or a combination thereof. As shown in FIG. 1, in some embodiments the MRI system 100 may include an imaging device 110, a processing device 120, a terminal device 130, a storage device 140, and a network 150. In some embodiments, the components in the MRI system 100 may be connected via the network 150 or directly. For example, the imaging device 110 may be connected to the terminal device 130 via the network 150. As another example, the imaging device 110 may be connected to the processing device 120 via the network 150 or directly. As yet another example, the imaging device 120 may be connected to the terminal device 130 via the network 150 or directly.

The imaging device 110 may be configured to scan an object or a portion of the object located in a detection region of the imaging device 110 and obtain scanning data (e.g., K-space data) related to the object or the portion of the object. In some embodiments, the object may include a biological object or a non-biological object. For example, the object may include a patient, an artificial object, etc. In some embodiments, the object may include a specific portion of the body, such as the head, the chest, the abdomen, or the like, or any combination thereof. In some embodiments, the object may include a specific organ, such as the heart, the esophagus, the trachea, the bronchus, the stomach, the gallbladder, the small intestine, the colon, the bladder, a ureter, the uterine, a tubal, or the like, or any combination thereof. In some embodiments, the object may include a region of interest (ROI), for example, a tumor, a nodule, etc.

In some embodiments, the imaging device 110 may include a magnetic resonance imaging (MRI) device.

The MRI device may include a magnet assembly, a gradient coil assembly, and a radio frequency (RF) coil assembly.

The magnet assembly may generate a first magnetic field (also referred to as a main magnetic field) configured to polarize an object to be detected. For example, the magnet assembly may include a permanent magnet, a superconducting electromagnet, a resistive electromagnet, etc.

The gradient coil assembly may generate a second magnetic field (also referred to as a gradient magnetic field). For example, the gradient coil assembly may include an X-gradient coil, a Y-gradient coil, and a Z-gradient coil. The gradient coil assembly may generate one or more magnetic field gradient pulses in an X-direction (Gx), a Y-direction (Gy), and a Z-direction (Gz) as shown in FIG. 1 on the main magnetic field to encode space information of the object. In some embodiments, the X-direction may be specified as a frequency encoding direction and the Y-direction may be specified as a phase encoding direction. In some embodiments, Gx may be configured for frequency encoding or signal readout, which may be often referred to as a frequency encoding gradient or a readout gradient. In some embodiments, Gy may be configured for phase encoding, which may be often referred to as a phase encoding gradient. In some embodiments, Gz may be configured for slicing (slice) selection to obtain two-dimensional (2D) K-space data. In some embodiments, Gz may be configured for phase encoding to obtain three-dimensional (3D) K-space data.

In the present disclosure, the X axis, the Y axis, and the Z axis shown in FIG. 1 may form an orthogonal coordinate system. The X axis and the Z axis shown in FIG. 1 may be horizontal, and the Y axis may be vertical. As illustrated, the positive X direction along the X axis may be from the right side to the left side of the imaging device 110 seen from the direction facing the front of the imaging device 110; the positive Y direction along the Y axis shown in FIG. 1 may be from the lower part to the upper part of the imaging device 110; the positive Z direction along the Z axis shown in FIG. 1 may refer to a direction in which the object is moved out of the scanning channel (or referred to as the bore) of the imaging device 110.

The RF coil assembly may include at least two RF coils. The RF coils may include one or more RF transmitting coils and/or one or more RF receiving coils. The

RF transmitting coil may transmit RF pulses to the object. In the synergistic effect of the main magnetic field/gradient field and the RF pulses, a magnetic resonance (MR) signal related to the object may be generated based on a pulse sequence. The RF receiving coil can obtain the MR signal from the object based on the pulse sequence. The MR signal may be processed using a transformation operation (e.g., Fourier transform) to fill K-space to obtain the K-space data.

The processing device 120 may process data and/or information obtained from the imaging device 110, the terminal device 130, and/or the storage device 140. For example, the processing device 120 may determine, based on undersampling K-space data of a plurality of phases of an object acquired by the imaging device 110, a first coil sensitivity map, determine, based on the first coil sensitivity map and the undersampling K-space data, a plurality of intermediate reconstruction images, and determine, based on the plurality of intermediate reconstruction images, an optimized dynamic image of the object through optimization processing. As another example, the processing device 120 may generate one or more image reconstruction models configured to generate the optimized dynamic image. In some embodiments, the processing device 120 may be a single server or a server group. The server group may be centralized or distributed. In some embodiments, the processing device 120 may be local or remote. For example, the processing device 120 may access information and/or data from the imaging device 110, the terminal device 130, and/or the storage device 140 via the network 150. As another example, the processing device 120 may be directly connected to the imaging device 110, the terminal device 130, and/or the storage device 140 to access the information and/or data. In some embodiments, the processing device 120 may be implemented on a cloud platform. For example, the cloud platform may include a private cloud, a public cloud, a hybrid cloud, a community cloud, a distributed cloud, an inter-cloud cloud, a multi-cloud, or the like, or any combination thereof. In some embodiments, the processing device 120 and the imaging device 110 may be integrated.

The terminal device 130 may include a mobile device 131, a tablet computer 132, a laptop computer 133, or the like, or any combination thereof. In some embodiments, the terminal device 130 may interact with other components in the MRI system 100 via the network 150. For example, the terminal device 130 may send one or more control instructions to the imaging device 110 via the network 150 to control the imaging device 110 to scan the object as the instructions. As another example, the terminal device 130 may receive the optimized dynamic image generated by the processing device 120 via the network 150 and display the optimized dynamic image for analysis and confirmation by an operator (e.g., a medical worker or a patient). In some embodiments, the mobile device 131 may include a smart home device, a wearable device, a mobile device, a virtual reality device, an augmented reality device, or the like, or any combination thereof.

In some embodiments, the terminal device 130 may be a portion of the processing device 120. In some embodiments, the terminal device 130 may be integrated with the processing device 120 as an operating console of the imaging device 110. For example, a user/operator (e.g., a doctor or a nurse) of the MRI system 100 may control the operation of the imaging device 110 via the operating console, for example, scanning the object, etc.

The storage device 140 may store data (e.g., the undersampling K-space data of the object, the reconstruction images, the optimized dynamic image, etc.), the instructions, and/or any other information. In some embodiments, the storage device 140 may store data obtained from the imaging device 110, the processing device 120, and/or the terminal device 130. For example, the storage device 140 may store the undersampling K-space data, the reconstruction images (e.g., intermediate reconstruction images, original reconstruction images, optimized dynamic images, etc.), etc., of the object obtained from the imaging device 110. In some embodiments, the storage device 140 may store the data and/or instructions that may be executed or used by the processing device 120 to perform the method for magnetic resonance imaging described herein.

In some embodiments, the storage device 140 may include a mass storage, a removable storage, a volatile read-and-write memory, a read-only memory (ROM), or the like, or any combination thereof. In some embodiments, the storage device 140 may be implemented via the cloud platform described in the present disclosure.

In some embodiments, the storage device 140 may be connected to the network 150 to achieve communication with one or more components (e.g., the processing device 120 or the terminal device 130) of the MRI system 100. The one or more components of the MRI system 100 may read the data or instructions in the storage device 140 via the network 150. In some embodiments, the storage device 140 may be a portion of the processing device 120, or may be independent and connected directly or indirectly to the processing device 120.

The network 150 may include any suitable network that can facilitate exchange of information and/or data of the MRI system 100. In some embodiments, the one or more components (e.g., the imaging device 110, the processing device 120, the terminal device 130, or the storage device 140) of the MRI system 100 may exchange information and/or data with the one or more components of the MRI system 100 via the network 150. For example, the processing device 120 may obtain the K-space data of the object from the imaging device 110 via the network 150. In some embodiments, the network 150 may include a public network (e.g., the Internet), a private network (e.g., a local area network (LAN), a wide area network (WAN)), a wired network (e.g., an Ethernet), a wireless network (e.g., an 802.11 network, a wireless Wi-Fi network, etc.), a cellular network (e.g., a Long Term Evolution (LTE) network), a frame relay network, a virtual private network (VPN), a satellite network, a telephone network, routers, hubs, server computers, or the like, or a combination thereof. In some embodiments, the network 150 may include one or more network access points. For example, the network 150 may include wired and/or wireless network access points, such as base stations and/or Internet exchange points, through which one or more components of the MRI system 100 may be connected to the network 150 to exchange data and/or information.

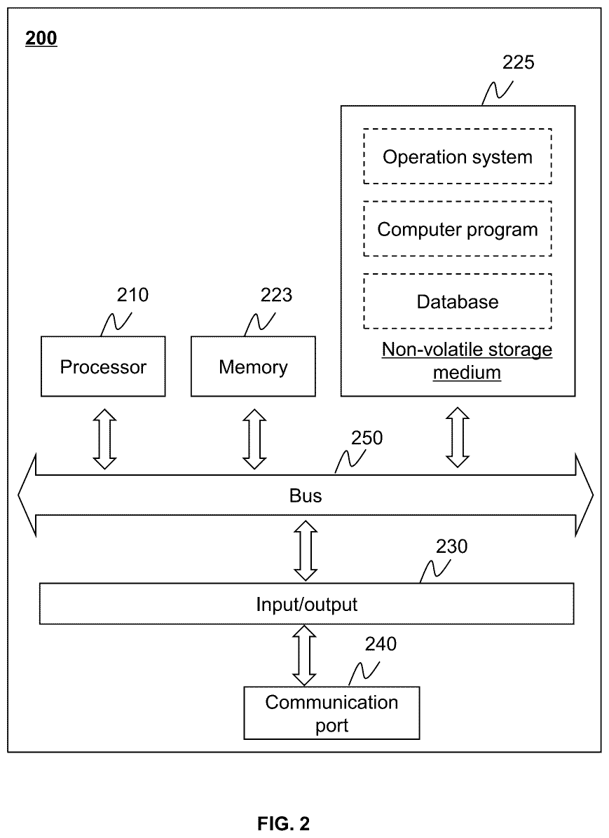

FIG. 2 is a schematic diagram illustrating exemplary software/hardware of a computing device according to some embodiments of the present disclosure. In some embodiments, a processing device (e.g., processing device 120) may be implemented by a computing device 200. In some embodiments, the computing device 200 may include a server, a personal computer, a laptop computer, a smartphone, a tablet computer, a smart mobile phone, etc.

As shown in FIG. 2, the computing device 200 may include a processor 210, a storage device, inputs/outputs 230, and a communication port 240. The storage device may include a non-volatile storage medium 225 and a memory 223. The processor 210, the storage device (e.g., the memory 223, the non-volatile storage medium 225), and the inputs/outputs 230 are connected via a system bus 250, and the communication port 240 is connected to the system bus 250 via the inputs/outputs 230.

The processor 210 may execute computer instructions (e.g., program code) and may perform functions of a processing device (e.g., the processing device 120) in accordance with the techniques described in the present disclosure. In some embodiments, the processor 210 may include one or more hardware processors, such as a microcontroller, microprocessor, etc. For illustrative purposes only, only one processor is described in computing device 200. However, it is noted that the computing device 200 may also include a plurality of processors. Operations and/or methods described in the present disclosure that are performed by a single processor may also be performed by a plurality of processors together or separately. For example, if a processor of the computing device 200 described in the present disclosure performs an operation A and an operation B, it should be appreciated that the operation A and the operation B may also be performed by two or more different processors of the computing device 200 jointly or separately (e.g., a first processor executing the operation A and a second processor executing the operation B, or the first processor and the second processor jointly executing the operations A and B).

The storage device may store data/information obtained from the imaging device 110, the terminal device 130, and/or any other component of the MRI system 100. For example, the non-volatile storage medium 225 may store an operating system, a computer program, and a database. The memory 223 may provide an environment for an operation of the operating system and computer programs in the non-volatile storage medium 225. The database may be configured to store data (e.g., undersampling K-space data, reconstructed image, intermediate K-space data, first coil sensitivity map, second coil sensitivity map, etc.) in the process of magnetic resonance image reconstruction. The processor 210 may execute the computer programs to implement a method for magnetic resonance imaging described herein.

The inputs/outputs 230 may be configured to exchange information between the processor 210 and an external device, such as the imaging device 110. In some embodiments, the input/outputs 230 may include an input device and an output device. The input device may include a keyboard, a mouse, a touch screen, a microphone, etc., or any combination thereof. The output device may include a display device, a speaker, a printer, a projector, or the like, or any combination thereof.

The communication port 240 may be configured to communicate with an external terminal (e.g., the imaging device 110) via a network connection. The connection may be a wired connection, a wireless connection, any connection that enables data transmission and/or reception, etc., or any combination thereof.

It should be understood that the descriptions of FIGS. 1 to 2 are only provided for the purpose of illustration and do not constitute a limitation to the present disclosure. For those skilled in the art, various changes and modifications can be made under the guidance of the present disclosure. Features, structures, manners and other characteristics of embodiments of the present disclosure can be combined in various ways to obtain other and/or alternative embodiments. However, such changes and modifications do not exceed the scope of the present disclosure. For example, the MRI system 100 may also include a display device configured to output an optimized dynamic image generated by the processing device 120. However, such changes and modifications do not depart from the scope of the present disclosure.

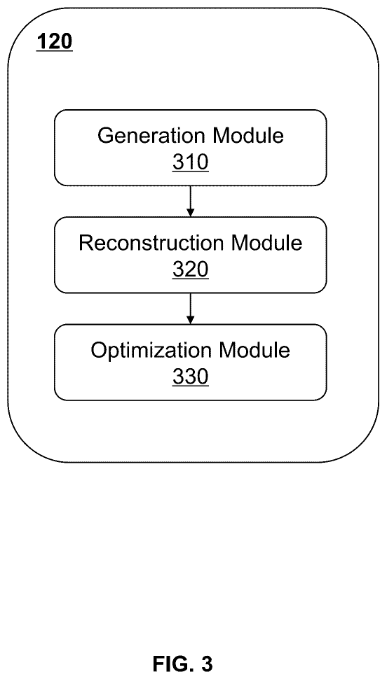

FIG. 3 is a schematic diagram illustrating an exemplary processing device according to some embodiments of the present disclosure.

As shown in FIG. 3, in some embodiments, the processing device 120 may include a generation module 310, a reconstruction module 320, and an optimization module 330. In some embodiments, one or more of the modules in the processing device 120 may be connected to each other. The connection may be wireless or wired.

The generation module 310 may be configured to determine, based on undersampling K-space data of a plurality of phases of an object, a first coil sensitivity map. In some embodiments, the generation module 310 may be configured to determine, based on the undersampling K-space data of the plurality of phases, intermediate K-space data, and determine, based on the intermediate K-space data, the first coil sensitivity map. In some embodiments, when an acquisition mode is a separately acquired mode, the generation module 310 may determine, based on the undersampling K-space data of the plurality of phases, a plurality of original reconstruction images, and determine the intermediate K-space data by combining (or averaging) the plurality of original reconstruction image. In some embodiments, when the acquisition mode is a concomitantly acquired mode, the generation module 310 may determine combined undersampling K-space data by averaging the undersampling K-space data of the plurality of phases, and determine, based on the combined undersampling K-space data, the intermediate K-space data.

The reconstruction module 320 may be configured to determine, based on the first coil sensitivity map and the undersampling K-space data, a plurality of intermediate reconstruction images. In some embodiments, the reconstruction module 320 may obtain the plurality of intermediate reconstruction images corresponding to the plurality of phases by performing initialization reconstruction on the plurality of phases respectively through a preset initialization reconstruction rule. In some embodiments, the reconstruction module 320 may determine the plurality of intermediate reconstruction images by performing, based on the undersampling K-space data of the first coil sensitivity map and the plurality of phases, initialization reconstruction using a sensitivity encoding (SENSE) algorithm.

The optimization module 330 may be configured to determine, based on the plurality of intermediate reconstruction images, an optimized dynamic image of the object through optimization process. In some embodiments, the optimization module 330 may obtain the optimized dynamic image of the object by inputting the plurality of intermediate reconstruction images into an image reconstruction model. The image reconstruction model may be a machine learning model.

It should be noted that the above descriptions of the processing device 120 are merely provided for the purpose of illustration, and not intended to limit the scope of the present disclosure. For those skilled in the art, various variations and modifications may be made according to the present disclosure. However, these variations and modifications do not depart from the scope of the present disclosure. For example, one or more modules of the processing device 120 may be omitted or integrated into a single module. As another example, the processing device 120 may include one or more additional modules, for example, a storage module for data storage, etc.

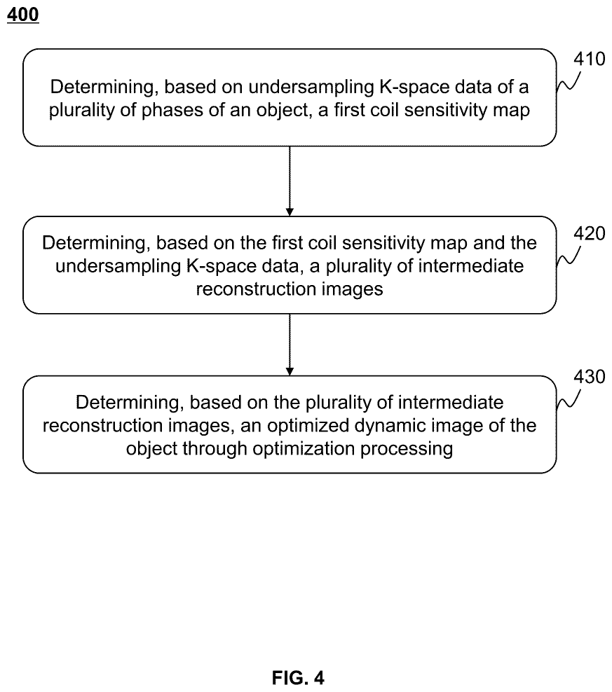

FIG. 4 is a flowchart illustrating an exemplary process for magnetic resonance imaging according to some embodiments of the present disclosure.

In some embodiments, process 400 may be performed by the MRI system 100. For example, the process 400 may be implemented as a set of instructions (e.g., an application) and stored, for example, outside the storage device 140 or the MRI system 100. The processing device 120 may execute the set of instructions and may be directed to perform process 400 when executing the set of instructions. The operations of the illustrated process 400 presented below are intended to be illustrative. Process 400 may be accomplished with one or more additional operations not described and/or without one or more of the operations discussed. In addition, the order of the operations of process 400 illustrated in FIG. 4 and described below is not intended to be limiting.

In 410, a first coil sensitivity map may be determined based on undersampling K-space data of a plurality of phases of an object. The undersampling K-space data may be obtained through Cartesian sampling. In some embodiments, operation 410 may be performed by the generation module 310.

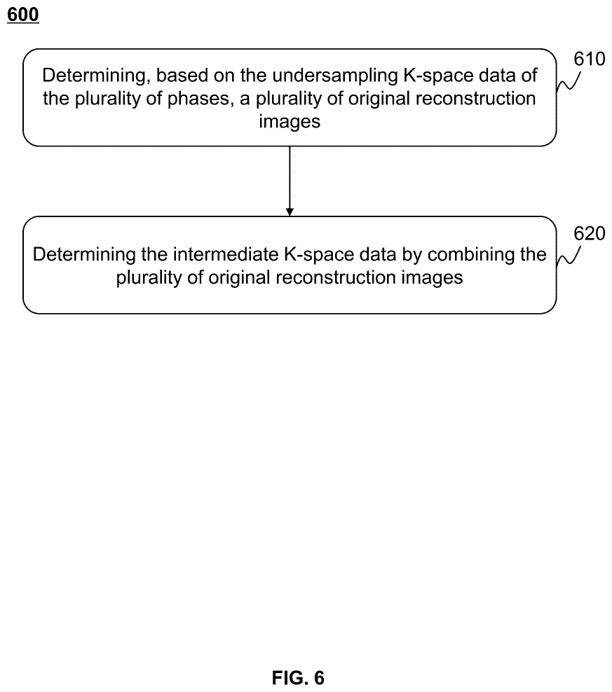

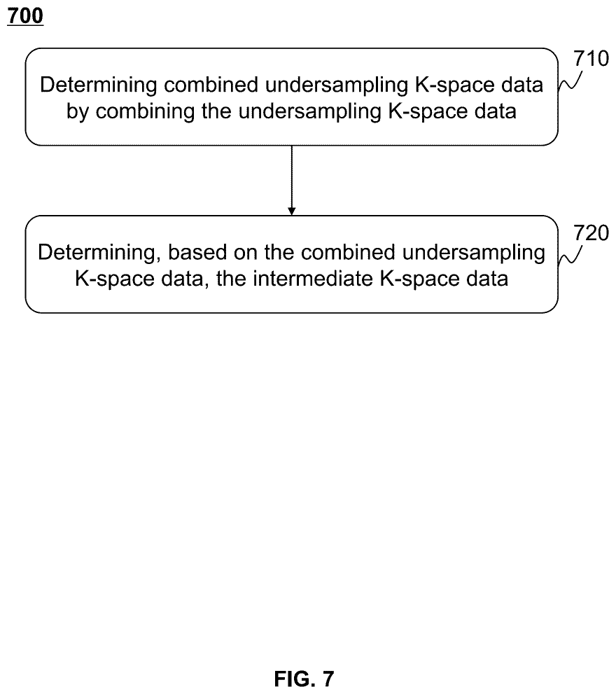

In some embodiments, the processing device 120 may determine intermediate K-space data by performing, based on the undersampling K-space data of the plurality of phases, average associated with the plurality of phases, and determining, based on the intermediate K-space data, the first coil sensitivity map. Details regarding determining the intermediate K-space data can be found elsewhere in the present disclosure (e.g., description in connection with FIG. 6 and FIG. 7).

There may be generally two forms of representation of images (i.e., a pixel domain (also referred to as an image domain or a time domain) and a frequency domain). The two forms may be converted to each other through Fourier transform and inverse Fourier transform. The Fourier transform is a transformation from the pixel domain to the frequency domain. Accordingly, the inverse Fourier transform is a transformation from the frequency domain to the pixel domain. K-space refers to a space used to store information in the frequency domain, that is, the frequency space of the Fourier transform (also referred to as the Fourier space). More specifically, in magnetic resonance imaging, a K-space refers to a filled space of original data of a magnetic resonance signal with spatial positioning encoding information, which may be a three-dimensional space or a planar two-dimensional space. A magnetic resonance image may be obtained based on different spatial frequencies each of which is arranged at a specific K-space position. The K-space data may be configured to characterize data converted from the magnetic resonance signal and may include 2D K-space data or 3D K-space data. For example, the 3D K-space data may be represented in a 3D coordinate axis system. The 3D coordinate axis system may include three coordinate axes corresponding to slicing selection, frequency encoding, and phase encoding. The 2D K-space data may be represented in a 2D coordinate axis system. The 2D coordinate axis system may include two coordinate axes corresponding to two of slicing selection, frequency encoding, and phase encoding.

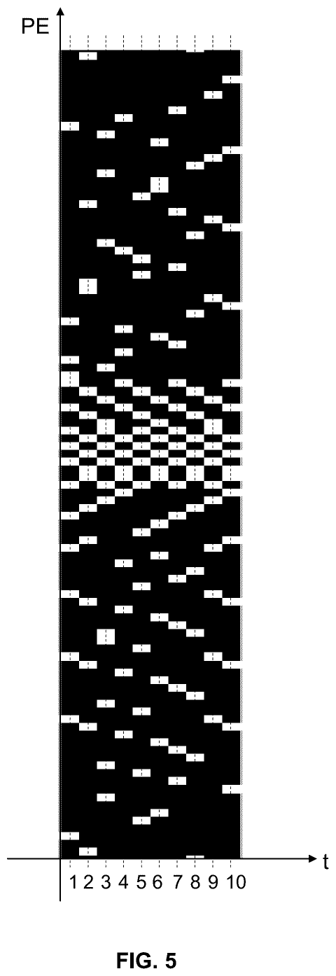

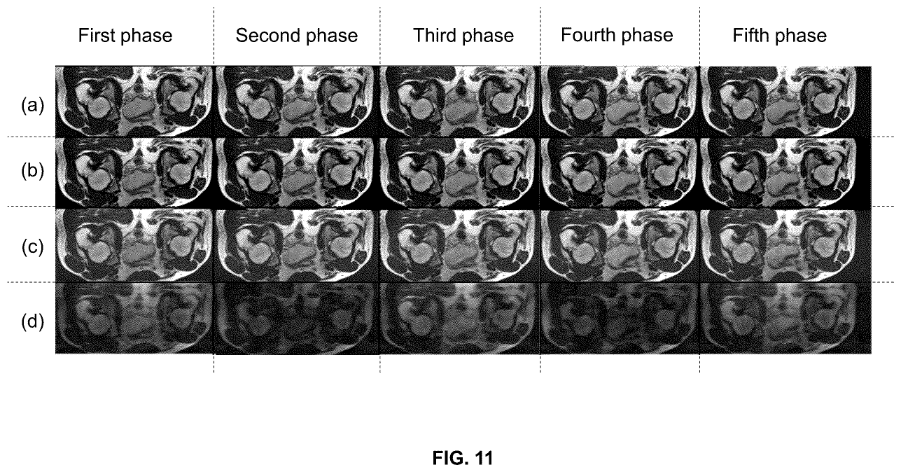

Merely by way of example, an MR device may perform space encoding (slicing selection, frequency encoding, and phase encoding) on MR signals with pulses of corresponding frequencies using three gradients during the imaging process, and obtain the K-space data by converting the obtained analog echo signals containing the spatial positioning encoding information into digital signals and filling the digital signals to the K-space. The MR signals of different frequencies, phases, and/or amplitudes may be obtained by resolving the K-space data through the inverse Fourier transform, and the MR digital signals of different frequencies, phases, and/or amplitudes may be distributed to corresponding pixels, and the magnetic resonance image of the object (i.e., a pixel domain image) may be obtained. In the K-space, the different frequencies and phases represent different spatial positions, and the amplitude represents the MR signal strength. For example, FIG. 5 shows the data mask in the K-space, and FIG. 11 shows the images in the image domain.

In conjunction with the above, during MRI, to improve the imaging speed, the scanning data may be usually obtained quickly through an undersampling technique. The undersampling technique refers to a way in which an MRI device performs sampling using a sampling frequency lower than an original signal bandwidth in the sampling process. In the embodiment of the present disclosure, the obtained K-space data of the plurality of phases of the object refers to the undersampling K-space data. The data obtained through the undersampling technique may be incomplete.

The undersampling K-space data of the plurality of phases refers to a plurality of sets of K-space data obtained by performing successive sampling on MR signals of the same object (e.g., the same target organ, the same patient, or the same region of interest, etc.) acquired by the MR device at multiple different time periods. Each time period may correspond to a phase. That is, each phase may generate an independent set of K-space data (e.g., undersampling 2D K-space data, or undersampling 3D K-space data), the plurality of phases may correspond to the plurality of sets of K-space data, and the plurality of sets of K-space data corresponding to the plurality of phases may be K-space datasets on the time series.

In some embodiments, the imaging device 110 (e.g., an MRI device) may obtain, by continuously performing undersampling on the object according to preset sampling trajectories, the undersampling K-space data of the plurality of phases of at least one slice of the object. The slice may be a two-dimensional slice (2D slice) or a three-dimensional imaging volume (3D volume). The slice may be any cross-section of the object, for example, a transverse section, a coronal plane, or a sagittal plane. For example, undersampling K-space data K_{A1}, K_{A2}, and K_{A3} of 3 phases of slice A may be obtained. As another example, the undersampling K-space data K_{A1}, K_{A2}, and K_{A3} of 3 phases of slice A, and undersampling K-space data K_{B1}, K_{B2}, and K_{B3} of 3 phases of slice B may be obtained. The phases corresponding to each slice may be the same or different. The imaging device 110 may send the obtained undersampling K-space data of the plurality of phases of the at least one slice of the object to the processing device 120 so that the processing device 120 may determine, by processing the obtained undersampling K-space data, the first coil sensitivity map.

A sampling trajectory reflects filling position of the acquired K-space data. In the sampling trajectory, a sampling density of a central region of the K-space may be greater than the sampling density of a peripheral region of the K-space.

For the convenience of description, in the present disclosure, the generating, based on the K-space data of the plurality of phases of a slice, the optimized dynamic image of the plurality of phases of the slice may be taken as an example. It is understood that if the K-space data of the plurality of phases of the plurality of slices are obtained separately, for each slice, the method provided in the present disclosure may be performed to generate an optimized dynamic image corresponding to the slice.

For one slice, each phase may correspond to one preset sampling trajectory, and the plurality of phases may correspond to different sampling trajectories. In some embodiments, the plurality of preset sampling trajectories corresponding to the plurality of phases may be interleaved in the K-space. That is, the preset sampling trajectories of phases may be distributed at different positions in the K-space. Accordingly, the undersampling K-space data of at least two neighboring phases of the plurality of phases may be obtained in an interleaved manner. That is, there may be a difference in the encoding positions of the undersampling K-space data of the at least two neighboring phases. The K-space data of different phases may represent sampling data at different positions in the K-space.

FIG. 5 is a schematic diagram illustrating an exemplary undersampling K-space data mask of a plurality of phases of a slice according to some embodiments of the present disclosure. The abscissa denotes the time t, the ordinate denotes the phase encoding PE, and the white rectangular box denotes the sampling data. For example, a white rectangular box represents K-space data at a sampling point in the k-space that is filled by sampling an MR signal that is acquired by an MR device via applying the corresponding phase encoding gradient during the MR scan of a subject. Each column in FIG. 5 represents a K-space data mask of one phase, and the K-space data of each phase may be obtained through undersampling using Cartesian sampling. The K-space data mask (i.e., 10 columns of white rectangular boxes in the figure) corresponding to 10 phases is shown. As shown in FIG. 5, the positions of the K-space data of each two neighboring phases in K-space are staggered and do not coincide. For example, the sampling data represented by the plurality of white rectangular boxes contained in column 1 and the sampling data represented by the plurality of white rectangular boxes contained in column 2 do not coincide and are interleaved with each other, i.e., corresponding to different phase encoding gradient.

The K-space data of the plurality of phases may be obtained in the interleaved manner of at least two neighboring phases, so that the sampling data of most positions (e.g., 90%, 95%, 98%, 100%, etc.) of the K-space may be obtained after the K-space data of the plurality of phases is fused.

The processing device 120 may obtain the undersampling K-space data of the plurality of phases of at least one slice of the object. In some embodiments, the processing device 120 may obtain the undersampling K-space data of the plurality of phases of at least one slice of the object from a picture archiving and communication system (PACS) or the storage device 140. For example, the processing device 120 may search the PACS for the undersampling K-space data of the plurality of phases of the object consecutively obtained at the at least one slice of the object based on identification information of the object. In the embodiment, there is no specific limitation on the manner in which the undersampling K-space data of the plurality of phases of the object is obtained.

In some embodiments, the undersampling K-space data may be obtained through Cartesian sampling. For one phase, the undersampling K-space data corresponding to the phase may be obtained through Cartesian sampling at the plurality of time points.

A coil sensitivity refers to a degree of an RF receiving coil responding to an input signal (e.g., an MR signal). The larger the value of the degree of response is, the better the ability of the RF receiving coil to detect the input signal may be, and the larger the coil sensitivity may be. The coil sensitivity map during MRI refers to a distribution map configured to describe sensitivity (i.e., the degree of response) of the RF receiving coil to MRI signals at different spatial positions. One same receiving coil may have different sensitivities to the magnetic resonance signals at different spatial positions. Different receiving coils may have different sensitivities to the magnetic resonance signals at the same spatial position. The receiving coil may have a relatively high sensitivity to a magnetic resonance signal close to the receiving coil and a relatively low sensitivity to a magnetic resonance signal far away from the receiving coil.

The first coil sensitivity map refers to a coil sensitivity map corresponding to complete K-space data. The plurality of phases may correspond to the same first coil sensitivity map. In some embodiments, the processing device 120 may determine, based on the undersampling K-space data of the plurality of phases, intermediate K-space data, and determine, based on the intermediate K-space data, the first coil sensitivity map.

The intermediate K-space data refers to complete K-space data obtained based on the undersampling K-space data of the plurality of phases.

According to different acquisition modes of the K-space data of the plurality of phases, the processing device 120 may determine the intermediate K-space data in different ways. For example, the acquisition modes may include a separately acquired mode and a concomitantly acquired mode.

The K-space data of the plurality of phases may also be referred to as imaging data. In some embodiments, when the imaging data is obtained, reference data may be additionally obtained from a central region of the K-space (e.g., a rectangular region or a circular region, etc., within a preset distance threshold or a preset distance from a center of the K-space). The reference data may be K-space data obtained by obtaining a number (e.g., 5, 8, 10, 12, 15, 18, 20, etc.) of phase encoding lines in the central region of the K-space. The separately acquired mode means that an independent K-space dataset (also referred to a reference dataset) is obtained by filling the reference data in an independent K-space. For example, when K-space data of three phases is obtained using the separately acquired mode, K-space data K1 corresponding to phase 1, K-space data K2 corresponding to phase 2, and K-space data K3 corresponding to phase 3 may be obtained. K1, K2, and K3 shares the same reference phase encoding line region (e.g., the same reference dataset).

The concomitantly acquired mode means that the reference data is contained in the undersampling K-space data corresponding to at least one of the plurality of phases (i.e., the reference data is filled in at least one K-space corresponding to the imaging data) instead of being an independent K-space dataset. For example, the K-space data of three phases may be obtained using the concomitantly acquired mode. 50 phase encoding lines may need to be obtained for each phase as the imaging data. In addition, additional 10 phase encoding lines in the central region of the K-space may need to be obtained as the reference data. For example, when K-space data corresponding to phase 1, phase 2, or phase 3 is acquired, in addition to acquiring the 50 phase encoding lines corresponding to a current phase, 10 phase encoding lines in the central region of the K-space may also need to be acquired, so that the K-space data including 60 phase encoding lines corresponding to the current phase may be formed. Merely by way of example, one of the 10 phase encoding lines in the central region of the K-space may be acquired after 5 of the 50 phase encoding lines are acquired each time. As another example, when the K-space data corresponding to phase 1 is obtained, 2 of the 10 phase encoding lines in the central region of the K-space may be acquired in addition to acquiring 50 phase encoding lines, so that K-space data K5 including 52 phase encoding lines corresponding to phase 1 may be formed. When the K-space data corresponding to phase 2 is acquired, 3 of the 10 phase encoding lines in the central region of the K-space may be acquired in addition to acquiring 50 phase encoding lines, so that K-space data K6 including 53 phase encoding lines corresponding to the phase 2 may be formed. When the K-space data corresponding to phase 3 is obtained, 5 of the 10 phase encoding lines in the central region of K-space may be acquired in addition to acquiring 50 phase encoding lines, so that K-space data K7 including 55 phase encoding lines corresponding to the phase 3 may be formed. The 2 phase encoding lines, 3 phase encoding lines, and 5 phase encoding lines of the 10 phase encoding lines in the central region of K-space may be different from each other.

In the separately acquired mode, since the data (reference data) in the central region of K-space is obtained separately, a phase reference line (i.e., auto calibration signals (ACS)) may be obtained. The phase reference line (e.g., obtained data corresponding to a number of phase encoding lines in the central region of K-space) may be configured to determine a coil sensitivity map (e.g., a first coil sensitivity map and a second coil sensitivity map).

In some embodiments, when the acquisition mode is the separately acquired mode, the processing device 120 may determine, based on the undersampling K-space data of the plurality of phases, a plurality of original reconstruction images, and determine the intermediate K-space data by combining (or averaging) the plurality of original reconstruction images. More descriptions regarding the determining the intermediate K-space data based on original reconstruction images may be found in FIG. 5 and the descriptions thereof.

In some embodiments, when the acquisition mode is the concomitantly acquired mode, the processing device 120 may determine combined undersampling K-space data by combining (or averaging) the undersampling K-space data of the plurality of phases, and determine, based on the combined undersampling K-space data, the intermediate K-space data. More descriptions regarding the determining, based on the combined undersampling K-space data, the intermediate K-space data may be found in FIG. 6 and the descriptions thereof.

In some embodiments, the processing device 120 may determine the first coil sensitivity map based on the intermediate K-space data using a preset algorithm.

For example, the first coil sensitivity map be determined using equation (1):

$$CSM = A / SO S ( A ) , \qquad ( 1 )$$

where CSM denotes the coil sensitivity map, A denotes the K-space data, and SOS denotes a root of a sum of squares of the K-space data.

When the first coil sensitivity map is determined using the equation (1), accordingly, CSM denotes the first coil sensitivity map, A denotes the intermediate K-space data, and SOS denotes a root of a sum of squares of the intermediate K-space data.

In 420, the processing device 120 may determine, based on the first coil sensitivity map and the undersampling K-space data, a plurality of intermediate reconstruction images. In some embodiments, operation 420 may be performed by the reconstruction module 320.

An intermediate reconstruction image refers to a visualization image obtained by performing image reconstruction on original obtained magnetic resonance signal data (e.g., undersampling K-space data corresponding to a phase).

The processing device 120 may obtain the plurality of reconstruction images (also referred to as intermediate reconstruction images) corresponding to the plurality of phases by performing initialization reconstruction on the undersampling K-space data of the plurality of phases, respectively through a preset initialization reconstruction rule. In the embodiments of the present disclosure, there is no specific limitation on the specific reconstruction mode (also referred to as the initialization reconstruction mode) for obtaining the plurality of intermediate reconstruction images. In some embodiments, the processing device 120 may determine the plurality of intermediate reconstruction images by performing, based on the first coil sensitivity map and the undersampling K-space data of the plurality of phases, initialization reconstruction using a sensitivity encoding (SENSE) algorithm. Each intermediate reconstruction image of the plurality of intermediate reconstruction images may correspond to one of the plurality of phases, that is, the obtained plurality of intermediate reconstruction images may form a dynamic MR image. For example, the processing device 120 may obtain the undersampling K-space data of 3 phases of a slice. The processing device 120 may determine an intermediate reconstruction image 1 based on the first coil sensitivity map and the undersampling K-space data of phase 1, and an intermediate reconstruction image 2 based on the first coil sensitivity map and the undersampling K-space data of phase 2, and determine an intermediate reconstruction image 3 based on the first coil sensitivity map and the undersampling K-space data of phase 3 using the SENSE algorithm. The intermediate reconstruction image 1, intermediate reconstruction image 2, and intermediate reconstruction image 3 may form ma dynamic MR image.

Exemplarily, for one of the plurality of phases, the processing device 120 may obtain filled K-space data (i.e., the complete K-space data of the current phase) of the phase by filling the undersampling K-space data of the phase based on the undersampling K-space data of other phases. Further, for one phase of the plurality of phases, the processing device 120 may obtain the intermediate reconstruction image corresponding to the phase by performing, based on the first coil sensitivity map and the full-sampling K-space data, image reconstruction, thereby obtaining the intermediate reconstruction image corresponding to the phase.

Before performing the image reconstruction, the processing device 120 may obtain the corresponding complete K-space data or K-space data whose density is greater than a preset threshold (e.g., 98%, 97%, 95%, 90%, etc.) by recovering the undersampling K-space data using a non-machine learning manner, thereby improving the quality of the reconstruction image. In some embodiments, the non-machine learning manner may include a phase correction and conjugate synthesis (PCCS) manner, a projection onto convex sets (POCS) manner, or the like, or a combination thereof.

Exemplarily, for one phase of the plurality of phases, the processing device 120 may recover the undersampling K-space data of the phase using the non-machine learning manner to complete the filling of the undersampling K-space data to obtain the complete K-space data of the phase, thereby obtaining the complete K-space data corresponding to the phase. In some embodiments, each set of undersampling K-space data corresponding to each phase of the plurality of phases may be recovered to obtain a plurality of sets of full-sampling k-space data and each set of complete K-space data may correspond to one phase. After data recovery is performed on the undersampling K-space data of each of the phases, the processing device 120 may obtain a multi-frame intermediate reconstruction image by performing reconstruction on the plurality of sets of complete K-space data corresponding to the plurality of phases using the first coil sensitivity map.

In the above reconstruction method, the complete K-space data may be obtained by recovering the undersampling K-space data of the plurality of phases using the non-machine learning manner, so that the data may be filled more accurately. Furthermore, the reconstruction may be performed on the complete K-space data of the plurality of phases using the first coil sensitivity map, so that the obtained information of the plurality of intermediate reconstruction images is more accurate and has better quality.

In 430, the processing device 120 may determine, based on the plurality of intermediate reconstruction images, an optimized dynamic image of the object through optimization processing. In some embodiments, operation 430 may be performed by the optimization module 330.

The optimized dynamic image refers to a plurality of target images with higher image quality obtained compared to the intermediate reconstruction images by performing reconstruction again on the plurality of intermediate reconstruction images in conjunction with information in the time direction. Each of the plurality of target images may correspond to one of the plurality of intermediate reconstruction images. That is, the optimized dynamic image may include the plurality of target images with time information. The count of the plurality of target images may be the same as the count of the plurality of intermediate reconstruction images.

Performing reconstruction again on the plurality of intermediate reconstruction images in conjunction with information in the time direction means a process in which the plurality of intermediate reconstruction images are arranged in a phase order (i.e., time order), and one of the arranged intermediate reconstruction images corresponding to a phase is optimized based on one or more intermediate reconstruction images of neighboring phases (e.g., for phase N, neighboring phases of the phase N may be phases N−1, N−2, N−3, N+1, N+2, N+3, or phases N−1 and N+1, or phases N−1, N−2, N+2, N+3) of the phase.

In some embodiments, the processing device 120 may obtain the optimized dynamic image of the object by inputting the plurality of intermediate reconstruction images into an image reconstruction model. The image reconstruction model may be a trained machine learning model.

In some embodiments, in addition to the plurality of intermediate reconstruction images, the input of the image reconstruction model may further include a second coil sensitivity map.

The second coil sensitivity map refers to a coil sensitivity map corresponding to the data in the central region of the K-space. The plurality of phases may correspond to the same second coil sensitivity map. As described above, the acquisition mode of the K-space data corresponding to the plurality of phases may include a separately acquired mode and a concomitantly acquired mode. According to the data different acquisition modes, the processing device 120 may determine the second coil sensitivity map in different ways.

When the acquisition mode is the separately acquired mode, since the ACS (e.g., the independent reference dataset) may be obtained, the processing device 120 may obtain the second coil sensitivity map directly based on the obtained ACS. For example, the second coil sensitivity map may be determined according to equation (1). At this time, the CSM denotes the second coil sensitivity map, A denotes the independent reference dataset (i.e., the K-space central region data obtained through the separately acquired mode), and SOS denotes a root of a sum of the squares of the independent reference dataset.

When the acquisition mode is a concomitantly acquired mode, the processing device 120 may determine the combined undersampling K-space data by combining (or averaging) the undersampling K-space data corresponding to the plurality of phases. More descriptions regarding the determining the combined undersampling K-space data may be found in FIG. 7. The processing device 120 may obtain the second coil sensitivity map based on the K-space central region data of the combined undersampling K-space data. For example, the processing device 120 may determine the second coil sensitivity map based on the K-space central region data of the combined undersampling K-space data according to equation (1), where CSM denotes the second coil sensitivity map, A denotes the K-space central region data of the combined undersampling K-space data, and SOS denotes a root of a sum of squares of the K-space central region data of the combined undersampling K-space data.

In some embodiments, the K-space central region data may not be additionally obtained (i.e., no reference data is obtained) in addition to obtaining the imaging data. In this case, the second coil sensitivity map may also be obtained using a manner similar to the first coil sensitivity map determination manner corresponding to the concomitant acquisition mode as described above.

In some embodiments, in addition to the plurality of intermediate reconstruction images, the input of the image reconstruction model may include the first coil sensitivity map. In some embodiments, in addition to the plurality of intermediate reconstruction images, the input of the image reconstruction model may further include the first coil sensitivity map and the second coil sensitivity map. In some embodiments, the input of the image reconstruction model may be determined to be the first coil sensitivity map and/or the second coil sensitivity map based on a cavity feature of the object. For example, if a cavity volume of the object is greater than a preset value, the second coil sensitivity map and the plurality of intermediate reconstruction images may be input to the image reconstruction model. If the cavity volume of the object is smaller than or equal to the preset value, the first coil sensitivity map and the plurality of intermediate reconstruction images may be input to the image reconstruction model.

In some embodiments, the processing device 120 may determine the cavity feature of the object using an image recognition model. An input of the image recognition model may include at least one of the intermediate reconstruction images, and an output of the image recognition model may include the cavity feature of the object corresponding to the intermediate reconstruction image. In some embodiments, the image recognition model may be obtained by training an initial convolutional neural network through first sample data. The first sample data may include a sample medical image. A label of the sample medical image may include a count of cavities and/or a cavity volume corresponding to the sample medical image. The first sample data may include a medical image in which a cavity exists and a medical image in which a cavity does not exist. The sample medical image may include an MRI image.

The second coil sensitivity map may be determined more quickly than the first coil sensitivity map. However, for a region with a relatively large count of cavities, the optimized dynamic image generated using the first coil sensitivity map may be prone to a problem of a low signal-to-noise ratio and loss of image quality. Therefore, the cavity feature of the object may be used to determine the input of the image reconstruction model. The first coil sensitivity map may be input into the image reconstruction model when the cavity volume of the object is relatively small, and the second coil sensitivity map may be input into the image reconstruction model when the cavity volume of the object is relatively large, so that the time and quality of the optimized dynamic image can be guaranteed at the same time.

In some embodiments, the image reconstruction model may include a first reconstruction unit (also referred to as a first image generator). The first reconstruction unit may include an iteration module. The iteration module may output a first dynamic image by performing a plurality of iterations on the plurality of intermediate reconstruction images. For the first iteration of the plurality of iterations, an input of the first iteration may include the plurality of intermediate reconstruction images.

Otherwise, the input of a next iteration may be an output of a previous iteration. The first dynamic image may include a plurality of first images. Each of the plurality of first images may correspond to one of the plurality of intermediate reconstruction images and one of the plurality of phases.

In some embodiments, parameters of the iteration module may include a plurality of attention matrices. Each of the attention matrices may correspond to one of the intermediate reconstruction images.

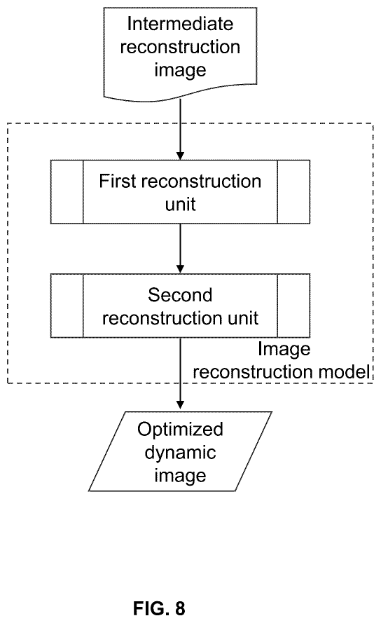

More descriptions regarding the iteration module outputting the first dynamic image may be found in the descriptions of FIGS. 8-10.

In some embodiments, the image reconstruction model may further include a second reconstruction unit (also referred to as a second image generator). The second reconstruction unit may output the optimized dynamic image by correcting at least one first image of the first dynamic image. The optimized dynamic image may include a plurality of second images. Each of the plurality of second images may correspond to one of the plurality of first images. An input of the second reconstruction unit may include the at least one first image, an intermediate reconstruction image corresponding to the at least one first image of the plurality of intermediate reconstruction images and a second coil sensitivity map. An output of the second reconstruction unit may include at least one corrected image corresponding to the at least one first image.

In some embodiments, the second reconstruction unit may be a two-dimensional structural portion, which may include a data fidelity layer and an image processing layer. The data fidelity layer and the image processing layer of the second reconstruction unit may be similar to those of the first reconstruction unit, which may not be repeated herein.

For one of the plurality of phases, the processing device 120 may input the first image of the phase output by the first reconstruction unit to the second reconstruction unit for correction. The second reconstruction unit may separately correct the first image corresponding to the phase in conjunction with the intermediate reconstruction image corresponding to the phase.

If the processing device 120 corrects all the first images, the processing device 120 may use the corrected images that are obtained as the plurality of second images. If the processing device 120 corrects a portion of the first images, the processing device 120 may use the corrected images that are obtained and first images that are not corrected as the plurality of second images.

The processing device 120 may determine the second dynamic image and the first dynamic image obtained through the second reconstruction unit as the optimized dynamic image of the object. For example, when the image reconstruction model only includes the first reconstruction unit, or only performs the iterative reconstruction of the first reconstruction unit, the processing device 120 may determine the first dynamic image obtained through the first reconstruction unit as the optimized dynamic image of the object. As another example, when the second dynamic image is obtained by correcting the at least one first image of the first dynamic image through the second reconstruction unit, the processing device 120 may determine the second dynamic image as the optimized dynamic image of the object.

Since the first reconstruction unit is a three-dimensional structural portion constructed based on a deep equilibrium models (DEQ) algorithm, the first reconstruction unit may combine (or average) information of neighboring phases during the reconstruction process. If too much information of the neighboring phases is used, an obtained reconstruction result may be inaccurate, that is, the obtained reconstruction result of the first dynamic image may be inaccurate. The at least one first image of the first dynamic image may be corrected by the second reconstruction unit, which may correct the reconstruction result to ensure the consistency between the image and the magnetic resonance undersampling image (i.e., the intermediate reconstruction image) corresponding to each phase in the optimized dynamic image, so that the obtained optimized dynamic image is closer to the real magnetic resonance full-sampling image.

In this embodiment, the secondary image reconstruction may be performed on the plurality of intermediate reconstruction images from overall and local dimensions through the three-dimensional structural portion (the first reconstruction unit) and the two-dimensional structural portion (the second reconstruction unit), which not only ensures the continuity of the reconstruction images in the time series, but also ensures the accuracy of the magnetic resonance undersampling image of each phase, thereby improving the reconstruction quality of the magnetic resonance image.

It should be noted that the above descriptions of process 400 are merely provided for the purposes of illustration, and not intended to limit the scope of the present disclosure. For those skilled in the art, various variations and modifications may be made according to the descriptions of the present disclosure. However, these variations and modifications do not depart from the scope of the present disclosure. In some embodiments, the process 400 may include one or more additional operations, or may omit one or more of the above operations. For example, the process 400 may include one or more additional operations for MRI imaging.

FIG. 6 is a schematic diagram illustrating an exemplary process for determining intermediate K-space data according to some embodiments of the present disclosure.

In some embodiments, process 600 may be performed by the MRI system 100. For example, the process 600 may be implemented as a set of instructions (e.g., an application) and stored, for example, outside the storage device 140 or the MRI system 100. The processing device 120 may execute the set of instructions and may be directed to perform process 600 when executing the set of instructions. The operations of the illustrated process 600 presented below are intended to be illustrative. Process 600 may be accomplished with one or more additional operations not described and/or without one or more of the operations discussed. In addition, the order of the operations of process 600 illustrated in FIG. 6 and described below is not intended to be limiting. In conjunction with the above, when an acquisition mode is a separately acquired mode, the processing device 120 may determine the intermediate K-space data through the following operations.

In 610, the processing device 120 may determine, based on the undersampling K-space data of the plurality of phases, a plurality of original reconstruction images. In some embodiments, operation 610 may be performed by the generation module 310.

In conjunction with the above, during the separately acquired mode, ACS may be obtained. The processing device 120 may determine a coil sensitivity map (i.e., a second coil sensitivity map) corresponding to a central region of the K-space based on the ACS, and further determine the plurality of original reconstruction images based on the undersampling K-space data of the plurality of phases and the second coil sensitivity map using the SENSE algorithm. In some embodiments, the processing device 120 may obtain the plurality of original reconstruction images corresponding to the plurality of phases by performing reconstruction on the undersampling K-space data of the plurality of phases respectively through a preset reconstruction algorithm. In some embodiments, the processing device 120 may determine the plurality of original reconstruction images by performing, based on the second coil sensitivity map and the undersampling K-space data of the plurality of phases, reconstruction on undersampling K-space data of each of the plurality of phases using the SENSE algorithm, respectively. Each of the plurality of original reconstruction images may correspond to one of the plurality of phases.

For example, when K-space data of three phases is obtained using the separately acquired mode, K-space data K1 corresponding to phase 1, K-space data K2 corresponding to phase 2, K-space data K3 corresponding to phase 3, and a reference dataset K4 may be obtained. The processing device 120 may determine the second coil sensitivity map according to the reference dataset K4 (e.g., according to equation (1)). The processing device 120 may determine an original reconstruction image 1 based on the second coil sensitivity map and the undersampling K-space data K1 of phase 1, determine an original reconstruction image 2 based on the second coil sensitivity map and the undersampling K-space data K2 of phase 2, and determine an original reconstruction image 3 based on the second coil sensitivity map and the undersampling K-space data K3 of phase 3 using the SENSE algorithm.

In 620, the processing device 120 may determine the intermediate K-space data based on the plurality of original reconstruction images. In some embodiments, operation 620 may be performed by the generation module 310.

In some embodiments, the processing device 120 may determine an intermediate image by weighting and combining (or averaging) the plurality of original reconstruction images corresponding to the plurality of phases, and determine the intermediate K-space data based on the intermediate image. For example, the processing device 120 may obtain the intermediate K-space data based on the intermediate image through Fourier transform.

In some embodiments, the processing device 120 may determine the intermediate image by averaging or weighted averaging the plurality of original reconstruction images. For example, the processing device 120 may obtain the intermediate image by averaging or weighted averaging pixel values of pixels corresponding to the same position in the plurality of original reconstruction images.

FIG. 7 is a schematic diagram illustrating an exemplary process for determining intermediate K-space data according to another embodiment of the present disclosure.

In some embodiments, process 700 may be executed by the MRI system 100. For example, the process 700 may be implemented as a set of instructions (e.g., an application) and stored in, for example, outside the storage device 140 or the MRI system 100. The processing device 120 may execute the set of instructions and may accordingly be directed to perform process 700 when executing the set of instructions. The operations of the illustrated process 700 presented below are intended to be illustrative. Process 700 may be accomplished with one or more additional operations not described and/or without one or more of the operations not discussed. Alternatively, the order of the operations of process 700 illustrated in FIG. 7 and described below is not intended to be limiting. In conjunction with the above, when an acquisition mode is a concomitantly acquired mode, the processing device 120 may determine the intermediate K-space data through the following operations.

In 710, the processing device 120 may determine combined undersampling K-space data by combining (or averaging) undersampling K-space data of a plurality of phases. In some embodiments, operation 710 may be performed by the generation module 310.

The processing device 120 may determine the combined undersampling K-space data by combining (or averaging) a plurality of sets of K-space data corresponding to the plurality of phases, that is, combining (or averaging) the plurality of sets of undersampling K-space data into one set of undersampling K-space data.

In some embodiments, the processing device 120 may determine the combined undersampling K-space data by performing weighted combining (or averaging) on the undersampling K-space data corresponding to the plurality of phases. Weights (also referred to as second weights) of the weighted combining (or averaging) may be related to sampling trajectories (i.e., preset sampling trajectories) of the plurality of phases.

In this embodiment, the weight may be presented in the form of a matrix or sequence. Each element of the matrix or sequence may correspond to a sampling point in the sampling trajectory. For example, if the preset sampling trajectory of each phase includes 100*100 sampling points, there may be a corresponding 100*100 weight matrix for each phase.

In some embodiments, a weight value of each element of the weight matrix or weight sequence may be inversely proportional to a count of sampling times at a sampling point corresponding to the element. The count of sampling times at the sampling point may be determined based on preset sampling trajectories of the plurality of phases. For example, for a certain sampling point, if preset sampling trajectories of three phases of the plurality of phases include the sampling point, the count of sampling times of the sampling point (also referred to as a sampling count of the sampling point) may be 3.

Exemplarily, if the sampling data of the target object includes K-space data of seven phases and a total of three sampling points A, B, and C, the preset sampling trajectory of phase 1 includes the sampling points A and B, the preset sampling trajectory of phase 2 includes the sampling point C, the preset sampling trajectories of phase 4 and phase 7 include the sampling point A, the preset sampling trajectory of phase 5 includes the sampling points B and C, and no data is obtained in phase 3 and phase 6, the sampling count of the sampling point A may be 3, and the weighting value corresponding to the sampling count of 3 may be ⅓; and both the sampling count of the sampling point B and the sampling count of the sampling point C may be 2, and the weighting value corresponding to the sampling count of 2 may be ½.

It is understood that for one same sampling point, the corresponding weight values in the weight matrices or sequences of different phases is the same, and the corresponding weight matrices in neighboring phases are different since the preset sampling trajectories of the neighboring phases are different.

By combining with the sampling trajectory, a more reasonable weight value may be determined, thereby obtaining the intermediate K-space data closer to the real situation. Because the undersampling K-space data of the plurality of phases are all for the same portion of the object, the processing device 120 may determine a product of the K-space data corresponding to each sampling point of the preset sampling trajectory of each phase and the corresponding weight value, and obtain the combined undersampling K-space data by summing the products corresponding to the same sampling point of the preset sampling trajectories of the plurality of phases.

In some embodiments, the processing device 120 may determine the second weight using a second weight determination model. An input of the second weight determination model may include the preset sampling trajectories of the plurality of phases, and an output of the second weight determination model may include second weights corresponding to the plurality of phases.

In 720, the processing device 120 may determine, based on the combined undersampling K-space data, the intermediate K-space data. In some embodiments, operation 720 may be performed by the generation module 310.

In some embodiments, the processing device 120 may determine, based on the combined undersampling K-space data, the intermediate K-space data through a Standard Protocol Items: Recommendations for Interventional Trials (SPIRIT) algorithm.

Exemplarily, the processing device 120 may determine a SPIRIT kernel based on K-space central region data of the combined undersampling K-space data, and perform iterative reconstruction based on the SPIRIT kernel and the combined undersampling K-space data through the SPIRIT algorithm. During each iteration, the SPIRIT may gradually fill in data points in an undersampling region (i.e., unacquired K-space data) using known K-space data (i.e., the undersampling K-space data corresponding to the plurality of phases) and the SPIRIT kernel. When all of the data points in the K-space is filled, complete K-space data may be obtained, and the processing device 120 may determine the complete K-space data to be the intermediate K-space data.

In some embodiments, the processing device 120 may obtain a second coil sensitivity map based on the K-space central region data of the combined undersampling K-space data. For example, the processing device 120 may determine the second coil sensitivity map based on the K-space central region data of the combined undersampling K-space data using the equation (1). The processing device 120 may determine the intermediate K-space data based on the second coil sensitivity map and the K-space data corresponding to the plurality of phases according to process 600.

In some embodiments, the K-space central region may not be additionally obtained (no reference data may be obtained) in addition to obtaining the imaging data. In this case, the intermediate K-space data may be obtained according to process 700.

It should be noted that the descriptions of process 600 and process 700 are merely provided for the purpose of illustration, and not intended to limit the scope of the present disclosure. For those skilled in the art, various variations and modifications may be made according to the descriptions in the present disclosure. However, these variations and modifications do not depart from the scope of the present disclosure.

In some embodiments, the image reconstruction model may include a first reconstruction unit.

An input of the first reconstruction unit may include a plurality of intermediate reconstruction images, and an output of the first reconstruction unit may include a first dynamic image.

In some embodiments, the first reconstruction unit may include an iteration module. The iteration module may be implemented based on Neural Network (NN), Convolutional Neural Network (CNN), or other machine learning techniques.

In some embodiments, the iteration module may include a data fidelity layer and one image processing layer. An input of the iteration module may be an input of the data fidelity layer. An output of the data fidelity layer may be an input of the image processing layer. An output of the image processing layer may be an output of the iteration module. The data fidelity layer may be configured to perform fidelity processing (also referred to as data consistency processing) on an input image. The data fidelity layer (also referred to as a data consistency (DC) layer) may be configured to ensure that there is a relatively high consistency between output data (i.e., input data of the image processing layer) of the data fidelity layer and real obtained data (i.e., undersampling K-space data of a plurality of phases). The image processing layer characterizing prior constraints may be configured to perform artificial intelligence (AI) processing on the input image to constrain the reconstruction process, and obtain the first dynamic image that satisfies a requirement by processing each image in conjunction with information in a time direction (information of images of neighboring phases).

Each of the data fidelity layer and image processing layer may be implemented based on deep neural networks such as Convolutional Neural Network (CNN), Deep Belief Network (DBN), Recurrent Neural Network (RNN), Long Short-Term Memory (LSTM), etc.

In some embodiments, the data fidelity layer may be NN, and the image processing layer may be CNN.

In some embodiments, the iteration module may obtain the first dynamic image by performing a plurality of iterations on the plurality of intermediate reconstruction images. Each iteration of the plurality of iterations may be performed by the iteration module.

In some embodiments, network parameters of the iteration module may be shared during the plurality of iterations. The network parameter sharing means that the network parameters of the iteration module are the same during each iteration.

In some embodiments, for each iteration of the plurality of iteration, when the iteration is a first iteration, an input of the iteration may include the plurality of intermediate reconstruction images, and when the iteration is not a first iteration, an input of the iteration may include an output of a previous iteration. An output of a last iteration of the plurality of iterations may be used as the first dynamic image.

In some embodiments, each iteration may include: inputting a plurality of images (i.e., the plurality of intermediate reconstruction images input to the iteration module, or the output of the previous iterative reconstruction) to the data fidelity layer of the iteration module, performing, by the data fidelity layer, fidelity processing on the plurality of images, and inputting a plurality of images output by the data fidelity layer into the image processing layer. The image processing layer may comprehensively determine output plurality of images based on the input plurality of images in conjunction with the information in the time direction (information of images of neighboring phases), and the processing result of the image processing layer may be used as the output of the iteration. For example, the image processing layer may optimize a current image based on one or more previous images of the current image, and/or one or more subsequent images of the current image. For example, in a first iteration, intermediate reconstruction images A1-D1 may be input into the iteration module, and the data fidelity layer may output images A2-D2 by performing fidelity processing on the intermediate reconstruction images A1-D1. Images A2-D2 may be input into the image processing layer. The image processing layer may determine an output image A3 corresponding to image A2 based on a feature of image B2 in conjunction with a feature of image A2, and determine an output image B3 corresponding to image B2 based on the feature of image A2 and a feature of image C2 in conjunction with a feature of image B2, determine an output image C3 corresponding to image C2 based on the feature of image B2 and a feature of image D2 in conjunction with a feature of image C2, and determine an output image D3 corresponding to image D2 based on the feature of image C2 in conjunction with a feature of image D2, that is, the image processing layer may output the multi-phase images A3-D3. After that, images A3-D3 may be used as an input of the iteration module in the second iteration. In this way, the first dynamic image may be finally obtained.

It should be noted that during the iterative reconstructions of the plurality of images, the plurality of images may always correspond to the plurality of phases, respectively.

In some embodiments, the input of the image processing layer may include an input and an output of the data fidelity layer, so that image processing layer may consider residual information based on the input and the output of the data fidelity layer.

In some embodiments, the plurality of iterative reconstructions may be realized based on Jacobian-free matrix computation.

In some embodiments, a count of the plurality of iterations may be a first count of times.

In some embodiments, the first count of times may be preset by the system or set by a user of the system.

In some embodiments, whether a plurality of images obtained after the plurality of iterations are performed satisfy a preset condition may be determined. If the preset condition is satisfied, the iteration process may be terminated, a count of executed iterations may be determined as the first count of times, and an image output by the iteration module from the last iteration may be used as the first dynamic image. If the preset condition is not satisfied, a next of iteration may be executed, and the image output by the iteration module from the last iteration may be used as an input of the iteration module for the next iteration. The preset condition may be set in advance, for example, the preset condition may include that the quality (e.g., signal-to-noise ratio, degree of artifacts, etc.) of the image output by the iteration module satisfies a certain condition. As another example, the preset condition may include that the output of the iteration module reaches a fixed point. More descriptions regarding the fixed point may be found below.

In some embodiments, the first count of times may be determined during training of the image reconstruction model. More descriptions may be found below.

In the embodiments of the present disclosure, a finite count of reconstructions may be performed by setting the first count of times and the result may be output in a timely manner, thereby improving the reconstruction efficiency.

In some embodiments, parameters of the iteration module may be obtained by training the iteration module. The parameters of the iteration module may include network parameters of the data fidelity layer and network parameters of the image processing layer.

In some embodiments, an optimization objective function when the iteration module is trained is expresses by equation (2) as followings:

$$\hat{x} = {arg min}_{x} \frac{η}{2} {‖ Ax - b ‖}_{2}^{2} + R ( x ) , \qquad ( 2 )$$

where x denotes the output image reconstructed by each iteration in the training process; \hat{x} denotes the output image of the iteration module when the training is completed; η denotes an attention matrix; A denotes a forward process of magnetic resonance undersampling; b denotes undersampling K-space data; and R(x) denotes an a priori constraint term or regularization term on the fast magnetic resonance imaging.

The iteration module may be trained by solving, based on any feasible algorithm, the above optimization objective function to obtain the parameters of the iteration module. In some embodiments, the optimization objective may be expanded based on a proximal gradient descent (PGD) algorithm according to equation (3):

$$P G D ( \hat{x} ) = \{ \begin{matrix}{x}^{k + \frac{1}{2}} = {x}^{k} - η {A}^{H} ( {Ax}^{k} - b ) \\ {x}^{k + 1} = Γ ( {x}^{k + \frac{1}{2}} ) = CNN ( {x}^{k + \frac{1}{2}} )\end{matrix} . \qquad ( 3 )$$

In the iteration module, the data fidelity layer may be configured to implement x^{k+1/2} of the expanded equation (3). The image processing layer may be configured to implement x^{k+1} of the expanded equation (3). The expanded equation (3) characterizes that the processing of the data fidelity layer is performed before the processing of the image processing layer is performed. However, in practical applications, the processing of the image processing layer may be performed before the processing of the data fidelity layer is performed.

In the expanded equation (3), k denotes a count of iterations, x^{k} denotes the image input into the data fidelity layer in the k iteration; x^{k+1/2} denotes the image output from the data fidelity layer in the k iteration, which is also the image input into the image processing layer at the same time; x^{k+1} denotes the image output from the image processing layer in the k iteration; Γ denotes the image processing layer, CNN denotes convolutional neural network, η denotes the attention matrix; A denotes the forward process of magnetic resonance undersampling, and H denotes the conjugate transpose of A.

The data fidelity layer may strengthen the data consistency between the input image and the output image through the imminent operator or data backfilling to realize the fidelity processing of the K-space data, which maximizes the preservation of the same K-space data between the input image and the output image of the data fidelity layer, ensures the consistency of the K-space data in the reconstruction process and enhances the stability and robustness of the whole image reconstruction model.

The image processing layer may extract the features of the input images of the plurality of phases. For the image of each phase, the processed image of each phase may be the output according to the features of the images of other phases (e.g., neighboring phases) in conjunction with the feature of the image of the phase to comprehensively determine the output first dynamic image, which comprehensively determines the output using the images of the plurality of phases, accelerates the convergence of the image reconstruction model, reduces reconstruction artifacts, and improves the quality of the reconstruction image at the same time.

In some embodiments, training the iteration module may specifically include the following operations.

An initial iteration module may be trained based on a large number of sample images of multiple phases with first labels. In some embodiments, the sample images of multiple phases may be input into the initial iteration module, a loss function may be constructed based on the plurality of images output by an initial iteration module and the first labels, and parameters of the initial iteration module may be iteratively updated based on the loss function until a termination condition is satisfied, and a trained iteration module may be obtained. The termination condition may include that the loss function is smaller than a threshold, converges, or a training cycle reaches a threshold. In some embodiments, the manner for iteratively updating the parameters of the model may include using a conventional model training algorithm such as a stochastic gradient descent algorithm, a projected gradient descent algorithm, etc.

In some embodiments, the sample images of multiple phases may include sample reconstruction images of a plurality of phases of at least one sample slice. Each sample reconstruction image may be obtained based on the undersampling K data of the corresponding phase. For example, the sample reconstruction image may be obtained based on the undersampling K data of the corresponding phase may be obtained using the method for generating the intermediate reconstruction image provided in the present disclosure, or may be obtained based on the undersampling K data of the corresponding phase using other reconstruction algorithms. The first label may include a plurality of MR fully-sampling images each of which corresponds to a sample reconstruction image of a phase of a sample slice. The MR fully-sampling image may be obtained by performing reconstruction on fully-sampling K-space data of the phase of the sample slice.

In some embodiments, the attention matrix n may be set according to human experience.

In some embodiments, the attention matrix n may be obtained through convolutional learning during training.

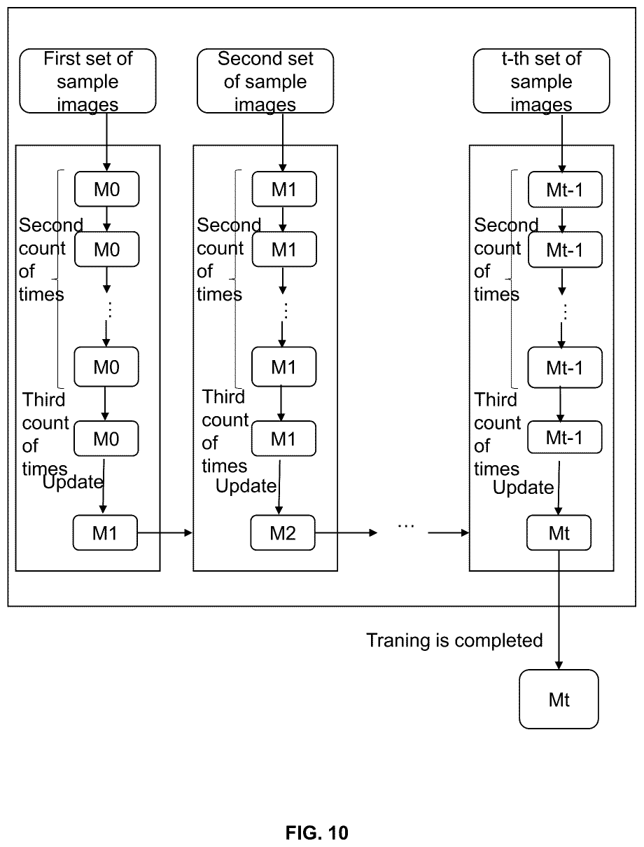

FIG. 10 is a schematic diagram illustrating an exemplary training process of an iteration module according to some embodiments of the present disclosure.

In some embodiments, in the training process of the iteration module, instead of updating parameters of the iteration module for each iteration, the training may be performed in a way that a same set of network parameters is shared for a plurality of iterations, which may include the following operation.

For each set of sample images of multiple phases, a computer device (e.g., the computing device 200) may input the sample images of multiple phases into the iteration module to execute the reconstruction process and count the executed iterations. In the training process, a second count of iterations may be executed. During the second count of iterations, the parameters of the iteration module may be kept unchanged. A third count of iterations may be executed. During the third count of iterations, the parameters of the iteration module may be updated. The second count of iterations may be performed again using an iteration module after network parameters are updated until the termination condition is satisfied

The second count and the third count may be preset. For example, the second count may be 10, and the third count may be 1. In this case, for every 10 iterations where the network parameters are not updated performed on the iteration module, one iteration where the network parameters are updated may need to be performed on the iteration module.

For example, as shown in FIG. 10, a first set of sample images of multiple phases may be input into an initial iteration module M0, and 10 (second count of times) iterations may be executed to obtain an output 1. Parameters of the initial iteration module M0 may be kept unchanged during the 10 iterations. The output 1 may be input into the initial iteration module M0, and 1 (third count of times) iteration may be performed to obtain an output 2. A first loss function value L11 may be determined based on the output 2 and first labels of the first set of sample images of multiple phases, and the parameters of the initial iteration module M0 may be updated based on the first loss function value, and an updated iteration module M1 may be obtained. A second set of sample images of multiple phases may be input into the iteration module M1, and 10 (second count of times) of iterations may be executed to obtain an output 3. Parameters of the iteration module M1 may be unchanged during the 10 iterations. The output 3 may be input into the iteration module M1, and 1 (third count of times) iteration may be performed to obtain an output 4. A first loss function value L12 may be determined based on the output 4 and first labels of the second set of sample images of multiple phases, and the parameters of the iteration module M1 may be updated based on the loss function value, and an iteration module M2 may be obtained. The iteration may be repeated until the iteration termination condition is met and a trained iteration module Mt may be obtained.

In some embodiments, during the third count of iterations for updating the parameters of the iteration module, a second loss function value may be determined according to an output of the data fidelity layer and undersampling K-space data of the set of sample images. The parameters of the iteration module may be updated based on the corresponding first loss function value and the second loss function value. The loss between the final output of the iteration module (the output of the image processing layer) and a gold standard (the first label) and the loss between the output of the data fidelity layer and the real undersampling K-space data may be simultaneously considered, which makes the objective of the training process be to simultaneously minimize the both loss and ensures that the iteration module can gradually optimize the final output at the same time when the consistency between the output of the data fidelity layer and the real obtained data is ensured, so that the final output is close to the real imaging result.

In some embodiments, the first loss function value and the second function value may be combined in a weighted manner to balance the importance in the training process. For example, a higher weight may be given to the first loss function value corresponding to the entire iteration module to ensure that the final output of the model is closer to the gold standard (the first label). As another example, a higher weight may be given to the second loss function value corresponding to the data fidelity layer to ensure physical or statistical consistency of the output.

In some embodiments, the second count of iterations during the model training may be used as a first count of iterations during model use.

In the embodiment of the present application, the termination condition may include that the output result of the iteration module reaches a fixed point, that is, the images output by the iteration module from two adjacent iterations are relatively similar. Exemplarily, whether a preset difference condition is satisfied may be determined based on the fixed point determined by a find fixed point (FFP) algorithm.

The FFP algorithm is expressed by equation (4) as follows:

$$\hat{k} = {arg min}_{k} \vert {x}^{k} - {x}^{k + 1} \vert , \qquad ( 4 )$$

where \hat{k} denotes a count of iterations when the iterative training terminates; x^{k} denotes an image output from a kth iteration; and x^{k+1} denotes an image output from a (k+1)^{th} iteration.

For example, the preset difference condition may include that an image similarity is greater than a preset threshold. Based on this, during the training process of the iteration module, the computer device may continuously obtain the image similarity between images output by the iteration module from two adjacent reconstruction processes (two adjacent iterations) to determine, based on the image similarity, whether the preset difference condition is met. For example, during the training process of the iteration module, 10 (second count of times) iterations T1-T10 may be executed with the same parameter of the iteration model, and 1 (third count of times) iteration T11 may be performed to update the parameter of the iteration module. The computer device may continuously obtain the image similarity between images output by the iteration module from two adjacent iterations of T1-T11. In the case where the image similarity is greater than or equal to the preset threshold, the preset difference condition is met, which indicates that a difference between a current reconstruction result and a previous reconstruction result is relatively small, so that it is considered that the current iteration module has converged, and the current result is recorded as the fixed point, at the same time, the subsequent iteration has little effect on the improvement of reconstruction effect by default, and the iterative training process may be terminated. On the contrary, in the case where the image similarity is smaller than the preset threshold, the preset difference condition is not met, which indicates that a difference the current reconstruction result and the previous reconstruction result is relatively large, so that the iteration module has not converged, and the iterative training process needs to continue to be performed.

In the embodiments of the present disclosure, iterative training of the iteration module may be performed through network parameter sharing, which reduces the data generated in the training process, reduces the memory occupation of the device carrying the model, solves the problem of memory explosion during training, realizes full training of the model under a limited memory condition, accelerates the convergence speed of the whole image reconstruction model, improves the convergence effect, and accordingly improves the quality of the image reconstructed based on the whole image reconstruction model.

In some embodiments, the parameters of the iteration module may further include a plurality of attention matrices (fidelity term coefficients). Each of the attention matrices may correspond to one of the plurality of intermediate reconstruction images. For an attention matrix corresponding to one intermediate reconstruction image, each element in the attention matrix may be a weight of a pixel in the intermediate reconstruction image. The iteration module may multiply, based on the attention matrix, a pixel value of each pixel of the intermediate reconstruction image by the corresponding weight when processing the intermediate reconstruction image, which makes the model pay more attention to information of a pixel with a relatively large weight using the attention matrix, so as to realize better reconstruction effect.

In some embodiments, the attention matrix may be obtained during the training of the iteration module.

FIG. 8 is a schematic diagram illustrating an exemplary image reconstruction model according to some embodiments of the present disclosure. A plurality of intermediate reconstruction images may be input into the image reconstruction model, and a first reconstruction unit may output a first dynamic image by processing the intermediate reconstruction images. The second reconstruction unit may output a second dynamic image by processing the first dynamic image. The second dynamic image may be used as an optimized dynamic image. More descriptions regarding the optimized dynamic image obtained by the image reconstruction model processing the intermediate reconstruction image may be found in the descriptions of other figures.

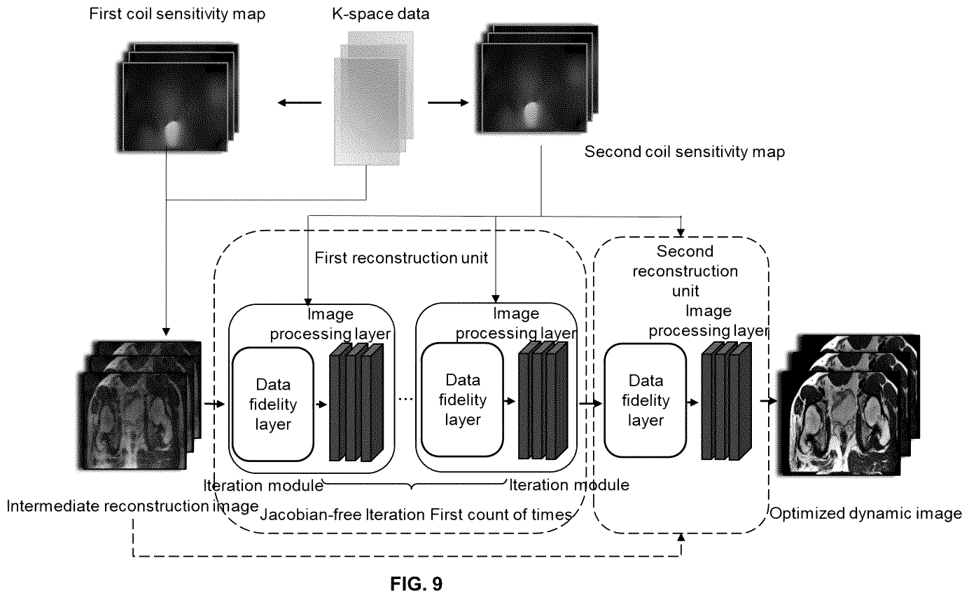

FIG. 9 is a schematic diagram illustrating an exemplary image reconstruction model according to some embodiments of the present disclosure. The processing device 120 may obtain undersampling K-space data of a plurality of phases. The processing device 120 may determine, based on the undersampling K-space data corresponding to the plurality of phases, a first coil sensitivity map and a second coil sensitivity map. The processing device 120 may determine, based on the undersampling K-space data corresponding to the plurality of phases and the first coil sensitivity map, the intermediate reconstruction images. The processing device 120 may input the intermediate reconstruction images and the second coil sensitivity map into the image reconstruction model. The image reconstruction model may include a first reconstruction unit and a second reconstruction unit. The first reconstruction unit may include an iteration module. The iteration module may include a data fidelity layer and an image processing layer. The data fidelity layer and the image processing layer of the iteration module may process input data of the image reconstruction model by performing a first count of iterations through a Jacobian-free matrix computation to obtain the first dynamic image. The second reconstruction unit may include a data fidelity layer and an image processing layer. The second reconstruction unit may obtain, by correcting the first dynamic image, a second dynamic image, and determine the second dynamic image as an optimized dynamic image. More descriptions regarding the structure of the image reconstruction model and the process for generating the optimized dynamic image based on the undersampling K-space data using the image reconstruction model may be found elsewhere in the present disclosure.

In some embodiments, the iteration module may perform the plurality of iterative reconstructions on the plurality of intermediate reconstruction images. In some embodiments, the first reconstruction unit includes a plurality of processing modules configured to perform the plurality of iterative reconstructions on the plurality of intermediate reconstruction images. The plurality of processing modules are connected sequentially, wherein an input of the first processing module includes the plurality of intermediate reconstructed images, an output of the last processing module includes the first dynamic image, and an input of the processing module that is not the first processing module includes an output of the previous processing module. Each of the plurality of iterative reconstructions is performed by one of the plurality of processing modules. For example, the first iterative reconstruction is performed by the first processing module, the second iterative reconstruction is performed by the second processing module, and so on.

In some embodiments, each processing module includes a data fidelity layer and an image processing layer. The data fidelity layer and the image processing layer of the processing module are similar to the data fidelity layer and the image processing layer of the iteration module, and are not described in detail here.

In some embodiments, structures and network parameters of the plurality of processing modules are the same.

FIG. 11 is a schematic diagram illustrating an exemplary reconstruction result according to some embodiments of the present disclosure.

As shown in FIG. 11, the K-space data of five phases may be illustrated as an example. Each phase includes an MR undersampling image (a), an initialization reconstructed MR image (b), an optimized MR image (c), and a fully-sampling MR image (d). The MR undersampling image refers to an image obtained by reconstructing directly based on the undersampling K-space data. The initialization reconstruction MR image refers to an intermediate reconstruction image obtained by reconstructing based on a first coil sensitivity map and undersampling K-space data. The optimized MR refers to an image determined by optimizing the intermediate reconstruction image. The fully-sampling MR image refers to an MR image obtained by reconstructing based on the fully-sampling K-space data of the corresponding phase. As shown in FIG. 11, the image quality of the MR undersampling image (a) is relatively poor. The image quality of the initialization reconstruction MR image (b) is improved compared to the MR undersampling image (a), and a difference between the optimized MR image (c) and the fully-sampling MR image (d) is relatively small. That is, in the MR reconstruction method provided by embodiments of the present disclosure, the MR image obtained based on the undersampling K-space data is closer to the MR image obtained by reconstruction based on the fully-sampling K-space data, and the image reconstruction quality is relatively good.

It should be noted that different embodiments may have different beneficial effects. In different embodiments, the possible beneficial effects may include any combination of one or more of the above, or any other possible beneficial effects that may be obtained.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements and amendments to the present disclosure. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, some features, structures, or features in the present disclosure of one or more embodiments may be appropriately combined.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for magnetic resonance imaging, implemented on a device including one or more processors and one or more storage devices, the method comprising:
determining intermediate K-space data by performing weighted average processing based on undersampling K-space data of a plurality of phases of an object, the undersampling K-space data being obtained through Cartesian sampling;
determining, based on the intermediate K-space data, a first coil sensitivity map; and
determining, based on the first coil sensitivity map, an optimized dynamic image of the object through optimization processing.

2. The method of claim 1, wherein the undersampling K-space data of each of the plurality of phases corresponds to a sampling trajectory, and the sampling trajectories corresponding to the plurality of phases are interleaved in K-space.

3. The method of claim 1, wherein the determining intermediate K-space data by performing, based on undersampling K-space data of a plurality of phases of an object, weighted average processing associated with the plurality of phases includes:
determining combined undersampling K-space data by averaging the undersampling K-space data of the plurality of phases; and
determining, based on the combined undersampling K-space data, the intermediate K-space data.

4. The method of claim 1, wherein the determining, based on the first coil sensitivity map, an optimized dynamic image of the object through optimization processing includes:
determining, based on the first coil sensitivity map and the undersampling K-space data, a plurality of intermediate reconstruction images, each of the plurality of intermediate reconstruction images corresponding to one of the plurality of phases; and
determining the optimized dynamic image of the object by inputting the plurality of intermediate reconstruction images into an image reconstruction model, the image reconstruction model being a machine learning model.

5. The method of claim 4, wherein the determining the optimized dynamic image of the object by inputting the plurality of intermediate reconstruction images into an image reconstruction model includes:
generating a first dynamic image by performing a plurality of iterative reconstructions on the plurality of intermediate reconstruction images, the first dynamic image including a plurality of first images each of which corresponds to one of the plurality of phases; and
generating the optimized dynamic image by separately correcting each of at least one of the plurality of first images based on the intermediate reconstruction image corresponding to the same phase as the first image.

6. The method of claim 5, wherein the generating a first dynamic image by performing a plurality of iterative reconstructions on the plurality of intermediate reconstruction images includes:
performing data consistency processing on the plurality of intermediate reconstruction images.

7. The method of claim 5, wherein the generating a first dynamic image by performing a plurality of iterative reconstructions on the plurality of intermediate reconstruction images includes:
for each of the plurality of first images, generating the first image based on information of the undersampling K-space data of a target phase of the plurality of phases corresponding to the target image and one or more neighboring phases of the target phase.

8. The method of claim 4, wherein an input of the image reconstruction model further includes at least one of the first coil sensitivity map or a second coil sensitivity map corresponding to data in a central region of K-space associated with the undersampling K-space data of the plurality of phases.

9. A method for magnetic resonance imaging, implemented on a device including one or more processors and one or more storage devices, the method comprising:
determining, based on undersampling K-space data of a plurality of phases of an object, a plurality of intermediate reconstruction images, each of the plurality of intermediate reconstruction images corresponding to one of the plurality of phases; and
inputting the plurality of intermediate reconstruction images into an image reconstruction model, the image reconstruction model being a machine learning model;
determining an optimized dynamic image of the object by performing, using the image reconstruction model, a plurality of iterative reconstructions on the plurality of intermediate reconstruction images.

10. The method of claim 9, wherein
the image reconstruction model includes a first reconstruction unit and a second reconstruction unit;
the first reconstruction unit includes a three-dimensional convolutional neural network;
the second reconstruction unit includes a two-dimensional convolutional neural network; and
an input of the second reconstruction unit includes an output of the first reconstruction unit.

11. The method of claim 9, wherein an input of the image reconstruction model further includes at least one of a first coil sensitivity map corresponding to complete K-space data associated with the undersampling K-space data of the plurality of phases or a second coil sensitivity map corresponding to data in a central region of K-space associated with the undersampling K-space data of the plurality of phases.

12. The method of claim 9, wherein the determining an optimized dynamic image of the object by performing, using the image reconstruction model, a plurality of iterative reconstructions on the plurality of intermediate reconstruction images includes:
generating a first dynamic image by performing the plurality of iterative reconstructions on the plurality of intermediate reconstruction images, the first dynamic image including a plurality of first images each of which corresponds to one of the plurality of phases; and
generating the optimized dynamic image based on the first dynamic image.

13. The method of claim 12, wherein the generating a first dynamic image by performing the plurality of iterative reconstructions on the plurality of intermediate reconstruction images includes:
performing data consistency processing on the plurality of intermediate reconstruction images.

14. The method of claim 12, wherein the generating a first dynamic image by performing the plurality of iterative reconstructions on the plurality of intermediate reconstruction images includes:
for each of the plurality of first images, generating the first image based on information of the undersampling K-space data of a target phase of the plurality of phases corresponding to the target image and one or more neighboring phases of the target phase.

15. The method of claim 12, wherein the generating the optimized dynamic image based on the first dynamic image includes:
generating the optimized dynamic image by separately correcting each of at least one of the plurality of first images based on the intermediate reconstruction image corresponding to the same phase as the first image.

16. An image reconstruction system, comprising:
an input layer configured to receive a plurality of intermediate reconstruction images generated based on undersampling K-space data of a plurality of phases of an object;
a first image generator configured to generate a first dynamic image of the object by performing a plurality of iterative reconstructions on the plurality of intermediate reconstruction images, the first dynamic image including a plurality of first images each of which corresponds to one of the plurality of phases, and; and
an output layer configured to output an optimized dynamic image of the object based on the first dynamic image.

17. The image reconstruction system of claim 16, wherein the first image generator includes:
a data consistency layer configured to perform data consistency processing on the plurality of intermediate reconstruction images.

18. The image reconstruction system of claim 17, wherein the first image generator includes:
an image processing layer configured to generate the first dynamic image in conjunction with information in a time direction, wherein for each of the plurality of first images, the image processing layer generates the first image based on information of the undersampling K-space data of a target phase of the plurality of phases corresponding to the target image and one or more neighboring phases of the target phase.

19. The image reconstruction system of claim 18, wherein for each of the plurality of iterative reconstructions, an input of the image processing layer includes an output and an input of the data consistency layer.

20. The image reconstruction system of claim 16, further comprising:
a second image generator configured to generate a second dynamic image by separately correcting each of at least one of the plurality of first images based on the intermediate reconstruction image corresponding to the same phase as the first image;
wherein the output layer is configured to output the second dynamic image as the optimized dynamic image.
